(19) United States
(12) Patent Application Publication
(10) Pub. No.: US 2020/0015663 A1
(43) Pub. Date: Jan. 16, 2020

(54) ENDOSCOPE SHEATH AND ENDOSCOPE SYSTEM

(71) Applicant: OLYMPUS CORPORATION, Tokyo (JP)

(72) Inventors: Kazutoshi KUMAGAI, Tokyo (JP); Naoya SUGIMOTO, Tokyo (JP); Yoshiro OKAZAKI, Tokyo (JP); Shunji TAKEI, Tokyo (JP)

(73) Assignee: OLYMPUS CORPORATION, Tokyo (JP)

(21) Appl. No.: 16/569,738

(22) Filed: Sep. 13, 2019

Related U.S. Application Data
(63) Continuation of application No. PCT/JP2017/011407, filed on Mar. 22, 2017.

(51) Int. Cl. A61B 1/00; A61B 1/12; A61B 1/005

(52) U.S. Cl. CPC A61B 1/00144; A61B 1/00073; A61B 1/005; A61B 1/12

(57) ABSTRACT

An endoscope sheath includes: a sheath body through which an endoscope channel is formed; a projecting portion extending in the longitudinal direction from the distal end of the sheath body and provided at a portion in the circumferential direction, on the radially outer side the endoscope channel; a nozzle from which the cleaning fluid is jetted radially inward of the sheath body and that is provided at the distal end portion of the projecting portion; and a fluid channel through which the cleaning fluid is supplied to the nozzle. At the distal end of the sheath body, the side opposite the nozzle with the endoscope channel therebetween in the radial direction is open.

This is a continuation of International Application PCT/JP2017/011407 which is hereby incorporated by reference herein in its entirety.

### TECHNICAL FIELD

The present invention relates to an endoscope sheath and an endoscope system.

### BACKGROUND ART

A known endoscope in the related art has, at the distal end face thereof, a nozzle through which cleaning liquid is jetted, and, by jetting the cleaning liquid onto observation-target tissue facing the distal end face, it is possible to wash off an adherent, such as blood, adhered to the observation-target tissue (for example, see Patent Literature 1). Another known endoscope sheath has a nozzle having an opening facing the distal end face of the endoscope, and, by jetting cleaning liquid from the nozzle onto the distal end face of the endoscope, it is possible to wash off an adherent adhered to an observation window at the distal end face (for example, see Patent Literature 2).

### CITATION LIST

### Patent Literature

{PTL 1} Japanese Unexamined Patent Application Publication No. 2003-153851
{PTL 2} Japanese Unexamined Patent Application Publication No. 2013-138790

### SUMMARY OF INVENTION

A first aspect of the present invention is an endoscope sheath including: a tubular sheath body having a penetrating endoscope channel, through which an endoscope is inserted, extending in the longitudinal direction; a projecting portion extending in the longitudinal direction from the distal end of the sheath body to a position beyond the distal end portion of the endoscope projecting from the distal end opening of the endoscope channel, the projecting portion being provided at a portion in the circumferential direction of the endoscope channel, on the outer side of the endoscope channel in the radial direction; a nozzle from which cleaning fluid is jetted radially inward of the sheath body, onto the distal end face of the distal end portion of the endoscope projecting from the distal end opening of the endoscope channel, the nozzle being provided in the distal end portion of the projecting portion; and a fluid channel through which the cleaning fluid is supplied to the nozzle, the fluid channel being provided in the sheath body and the projecting portion so as to extend in the longitudinal direction. The side opposite the nozzle with the endoscope channel therebetween in the radial direction is open at the distal end of the sheath body.

### BRIEF DESCRIPTION OF DRAWINGS

FIG. 1 shows the overall configuration of an endoscope sheath and an endoscope system provided with the same according to an embodiment of the present invention.

FIG. 2A is a side view of a distal end portion of the endoscope sheath, showing how to clean an observation window in the endoscope system in FIG. 1.

FIG. 2B is a side view of the distal end portion of the endoscope sheath, showing how to clean an observation area of an observation-target tissue by using the endoscope system in FIG. 1.

FIG. 3 is a cross section of the endoscope sheath taken along line I-I in FIG. 1.

FIG. 4 is a cross section of the endoscope sheath taken along line II-II in FIG. 1, showing the configuration of a fixing portion.

FIG. 5 shows how to use the endoscope system in FIG. 1.

FIG. 6A is a side view of the distal end portion of the endoscope sheath, showing how to clean the observation window when an oblique-viewing endoscope is used.

FIG. 6B is a side view of a distal end portion of the endoscope sheath, showing how to clean the observation area of the observation-target tissue when an oblique-viewing endoscope is used.

FIG. 7 shows the overall configuration of a modification of the endoscope system in FIG. 1.

FIG. 8 is a side view of a distal end portion of the endoscope sheath showing a modification of the nozzle shape.

FIG. 9 is a perspective view of the distal end portion of another modification of the endoscope system in FIG. 1.

FIG. 10A is a side view of the distal end portion of the endoscope sheath, showing the arrangement of multiple nozzles.

FIG. 10B is a cross section of the distal end portion of the endoscope sheath, showing another arrangement of the multiple of nozzles.

### DESCRIPTION OF EMBODIMENTS

An endoscope sheath 1 and an endoscope system 100 according to an embodiment of the present invention will be described below with reference to the drawings.

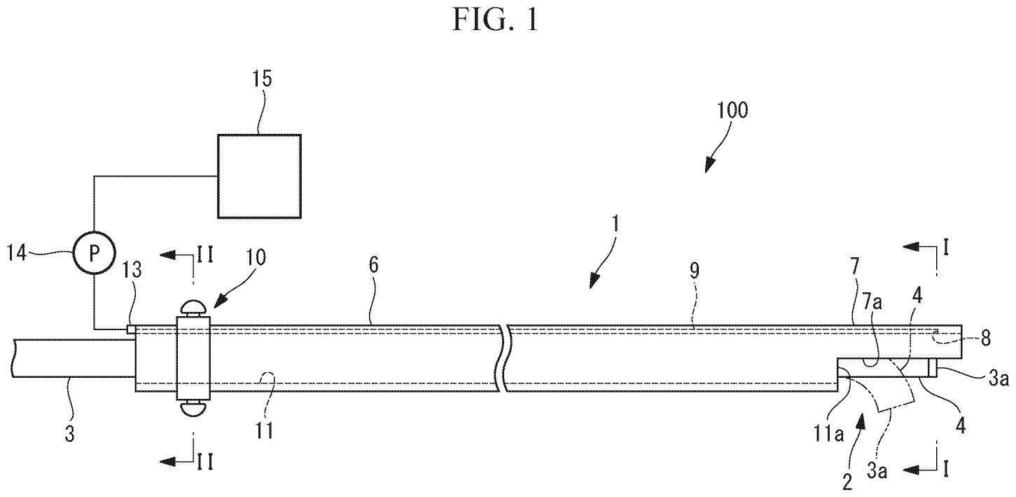

As shown in FIG. 1, the endoscope system 100 according to this embodiment includes an endoscope 2 and the endoscope sheath 1 having an endoscope channel 11 through which the endoscope 2 can be inserted.

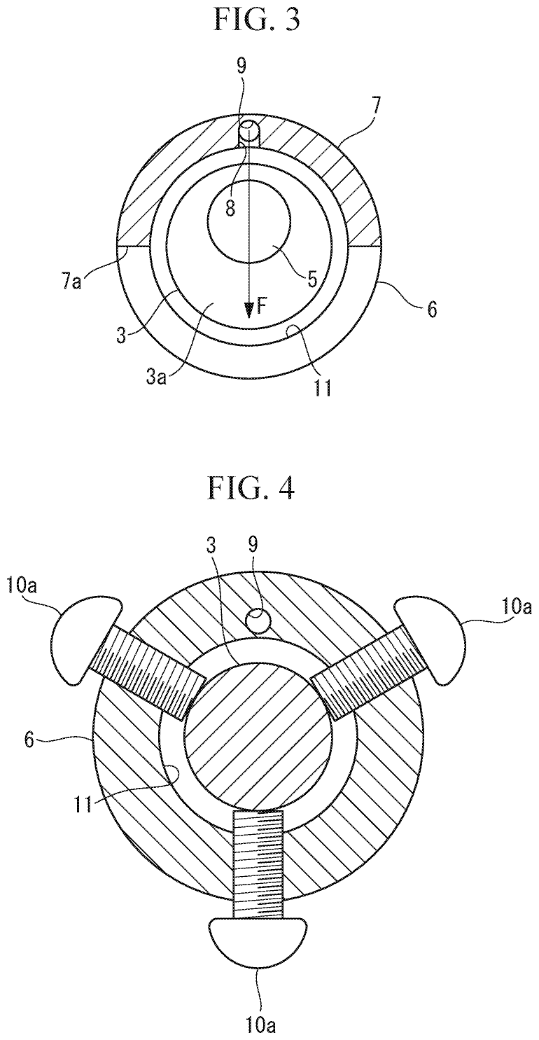

The endoscope 2 has a long, flexible, and small-diameter insertion section 3 that can be inserted into the pericardial space, and a bending portion 4 that is provided at the distal end portion of the insertion section 3 and that is bendable in at least one direction. The endoscope 2 is a forward-viewing endoscope, which has an observation window 5 (see FIG. 3) in a distal end face 3a perpendicular to the longitudinal axis of the insertion section 3.

The endoscope 2 has an objective lens (not shown) and an image-capturing device (not shown) at the distal end portion. The light entering the observation window 5 is made to form an image by the objective lens, and the thus-formed image is captured by the image-capturing device. Alternatively, a configuration in which a fiber bundle extending in the longitudinal direction is provided inside the endoscope 2, and an image formed by the objective lens is optically transmitted through the fiber bundle to an image-capturing device provided at the proximal end portion of the endoscope 2 is also possible. The image signal obtained by the image-capturing device is electrically transmitted to the image processor, where an endoscope image is generated, and the endoscope image is displayed on a monitor.

The endoscope 2 may be an optical scanning endoscope, in which a laser beam is two-dimensionally scanned over a subject, reflected light of the laser beam reflected from the subject is received through the observation window 5, and a two-dimensional image of the subject is generated on the basis of the scanning position of the laser beam and the intensity of the reflected light.

The endoscope sheath 1 includes a cylindrical sheath body 6 that has a longitudinal axis and that is open at both ends, a projecting portion 7 that is continuous with the distal end of the sheath body 6 and that projects from the distal end of the sheath body 6 in the longitudinal direction, a nozzle 8 that is provided at the distal end portion of the projecting portion 7 and from which cleaning fluid F is jetted, a fluid channel 9 through which the cleaning fluid F is supplied to the nozzle 8, and a fixing portion (position restricting portion, rotation restricting portion) 10 that fixes the position and orientation of the insertion section 3 disposed inside the endoscope channel 11.

The sheath body 6 is flexible and thus can bend in conformance to the shape of tissue in a living body. The sheath body 6 has, inside thereof, the endoscope channel 11 penetrating along the longitudinal axis from the proximal end opening in the proximal end face of the sheath body 6 to the distal end opening 11a in the distal end face. The endoscope channel 11 has a larger inside diameter than the outside diameter of the insertion section 3, and thus, the insertion section 3 can move in the longitudinal direction and can rotate about the longitudinal axis in the endoscope channel 11.

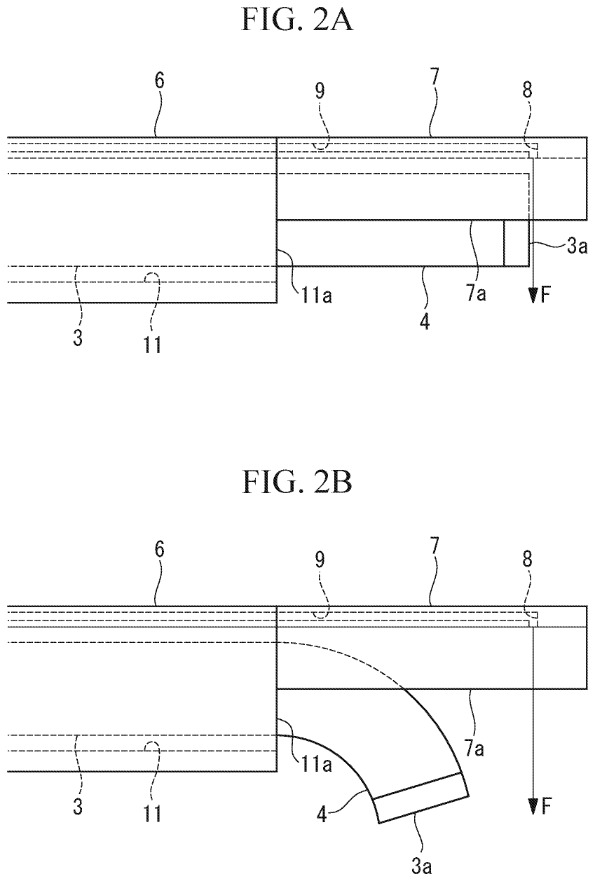

The projecting portion 7 has a substantially partially cylindrical shape having, at a portion in the circumferential direction, a cutaway portion 7a removed in the longitudinal direction. The projecting portion 7 projects in the longitudinal direction of the sheath body 6 from a portion, in the circumferential direction, of the ring-shaped distal end face of the sheath body 6 surrounding the distal end opening 11a and is located radially outside of the endoscope channel 11 when the sheath body 6 is viewed in the longitudinal direction from the distal-end side. As shown in FIG. 2A, the projecting portion 7 has a length reaching a position beyond the distal end face 3a of the insertion section 3 in a state in which the bending portion 4 projects from the distal end opening 11a.

The width of the cutaway portion 7a in the radial direction of the sheath body 6 is larger than the diameter of the bending portion 4. This enables the distal end face 3a of the insertion section 3 to project in the radial direction from the cutaway portion 7a when the bending portion 4 is bent toward the cutaway portion 7a inside the projecting portion 7, as shown by a two-dot chain line in FIG. 1. Although the illustrated projecting portion 7 has a substantially semi-cylindrical shape in which substantially half of the circumference is removed over the entire length in the longitudinal direction, the length and width of the cutaway portion 7a may be changed as appropriate.

Furthermore, the projecting portion 7 has sufficient rigidity to resist the pressing force toward the heart applied from the pericardium inside the pericardial space and to maintain a substantially straight shape.

As shown in FIG. 3, the nozzle 8 is open in the inner surface of the projecting portion 7, at a position opposite the cutaway portion 7a with the endoscope channel 11 therebetween, in the radial direction of the endoscope channel 11. The nozzle 8 has a cylindrical inner surface extending perpendicular to the longitudinal direction of the endoscope channel 11 toward the central axis of the endoscope channel 11 and jets the cleaning fluid F radially inward of the endoscope channel 11 toward the cutaway portion 7a. Furthermore, as shown in FIG. 2A, the nozzle 8 is provided at a position adjacent to the distal end face 3a of the insertion section 3 in the radial direction, in a state in which the bending portion 4 projects from the distal end opening 11a.

Accordingly, as shown in FIG. 2A, by providing the distal end face 3a of the insertion section 3 at a position adjacent to the nozzle 8 in the radial direction, it is possible to spray the cleaning fluid F jetted from the nozzle 8 onto the distal end face 3a to clean the observation window 5. Furthermore, from the state in FIG. 2A, by bending the bending portion 4 toward the cutaway portion 7a to create, on the distal-end side of the bending portion 4, a space for the cleaning fluid F to pass, as shown in FIG. 2B, it is possible to spray, through the endoscope channel 11 and the cutaway portion 7a, the cleaning fluid F jetted from the nozzle 8 onto the living body tissue located beside the sheath body 6 to clean the living body tissue.

The fluid channel 9 is formed in the side wall of the sheath body 6 and the projecting portion 7 so as to extend in the longitudinal direction from a supply port 13 provided at the proximal end portion of the sheath body 6 to the nozzle 8. The supply port 13 is connected, via a pump 14, to a fluid source 15 storing the cleaning fluid F, and the cleaning fluid F is supplied from the fluid source 15 to the nozzle 8 through the supply port 13 and the fluid channel 9 by actuating the pump 14. The cleaning fluid F may be either liquid, such as physiological saline, or gas that blows off the adherent, such as blood, on the living body tissue and the observation window 5.

The fixing portion 10 fixes an intermediate portion, in the longitudinal direction, of the insertion section 3 inserted through the endoscope channel 11 to the sheath body 6. As shown in FIG. 4, this fixing portion 10 includes, for example, a plurality of fixing screws 10a that are provided at intervals in the circumferential direction of the sheath body 6 and that penetrate through the side wall of the sheath body 6 in the radial direction from the outside of the sheath body 6 to the inside of the endoscope channel 11. By tightening the fixing screws 10a, it is possible to restrict the position of the insertion section 3 in the longitudinal direction and the rotation thereof relative to the sheath body 6, such that the insertion section 3 does not move forward or backward or rotate relative to the sheath body 6. The distal end portions of the fixing screws 10a, which are in contact with the insertion section 3, may be covered with an elastic material, such as rubber, to protect the outer surface of the insertion section 3.

Next, the advantages of the thus-configured endoscope system 100 will be described.

When a heart A is observed by using the endoscope system 100 according to this embodiment, first, the insertion section 3 of the endoscope 2 is inserted through the endoscope channel 11 in the sheath body 6. Then, as shown in FIG. 2A, in a state in which the bending portion 4 extends straight, the position of the insertion section 3 in the longitudinal direction inside the endoscope channel 11 is determined such that the distal end face 3a of the insertion section 3 is adjacent to the nozzle 8 in the radial direction. Furthermore, as shown in FIG. 2B, the orientation of the insertion section 3 about the longitudinal axis in the endoscope channel 11 is determined such that the bending portion 4 is bent toward the side opposite to the side where the nozzle 8 is provided (i.e., toward the side of the cutaway portion 7a). Next, the insertion section 3 is fixed to the sheath body 6 with the fixing portion 10, in accordance with the determined position and orientation.

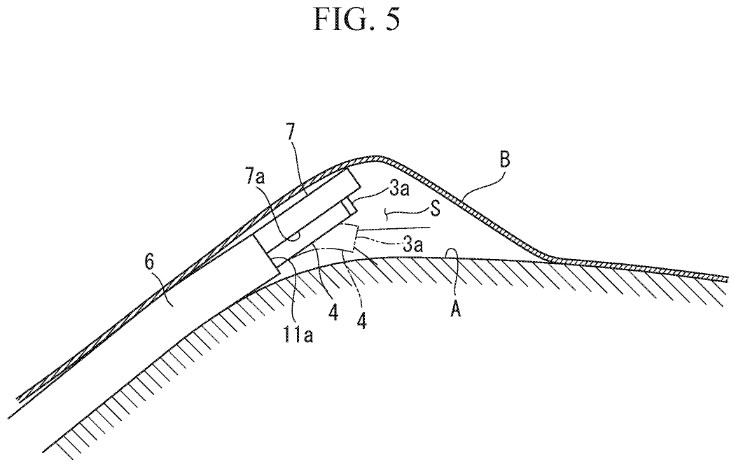

Next, the sheath body 6 and the insertion section 3 integrated by the fixing portion 10 are inserted into the pericardial space through a tubular trocar preliminarily disposed so as to extend from the outside of the body to the interior of the pericardial space. As shown in FIG. 5, by disposing, inside the pericardial space, the sheath body 6 such that the projecting portion 7 is located on the pericardium B side, the pericardium B is lifted and held at a position away from the heart A by the projecting portion 7 having high rigidity, and thus, a space S is created around the distal end face 3a of the insertion section 3. By bending the bending portion 4 toward the cutaway portion 7a in this state, it is possible to dispose the observation window 5 in the distal end face 3a of the insertion section 3 so as to face the heart A, which is the observation-target tissue, to observe the heart A from above.

When an adherent, such as blood, is adhered to the observation window 5 during the observation in the pericardial space, requiring cleaning of the observation window 5, the operator returns the bending portion 4 to a straight state, as shown by a solid line in FIG. 5, and actuates the pump 14. As a result, the cleaning fluid F is jetted from the nozzle 8 onto the distal end face 3a of the insertion section 3, which is located in front of the nozzle 8, and the observation window 5 is cleaned with the cleaning fluid F.

Furthermore, when an adherent, such as blood, is adhered to the observation area of the heart A, requiring cleaning of the observation area during the observation inside the pericardial space, the operator retracts the bending portion 4 from the path of the cleaning fluid F jetted from the nozzle 8 by bending the bending portion 4 toward the cutaway portion 7a, as shown by a two-dot chain line in FIG. 5, and actuates the pump 14. As a result, the cleaning fluid F jetted from the nozzle 8 is jetted onto the observation area of the heart A through the cutaway portion 7a without being blocked by the distal end portion of the insertion section 3, and the observation area is cleaned with the cleaning fluid F. At this time, because the observation window 5 is facing the heart A, it is possible to observe, with the endoscope 2, the observation area of the heart A being cleaned.

As has been described above, in this embodiment, the nozzle 8 is provided at a position aligned with the distal end face 3a of the insertion section 3 in the radial direction, in a state in which the bending portion 4 projects from the distal end opening 11a of the endoscope channel 11. Moreover, because the cutaway portion 7a is provided on the side opposite the nozzle 8 with the endoscope channel 11 therebetween in the radial direction, the heart-A-side portion of the distal end of the sheath body 6 is open. This leads to an advantage in that it is possible to efficiently clean the observation area, which is observed by bending the bending portion 4 toward the heart A, with the cleaning fluid F jetted from the nozzle 8. There is also an advantage in that it is possible to switch the cleaning target between the observation window 5 and the observation area of the heart A by a simple operation of bringing the bending portion 4 into a straight state or a bent state.

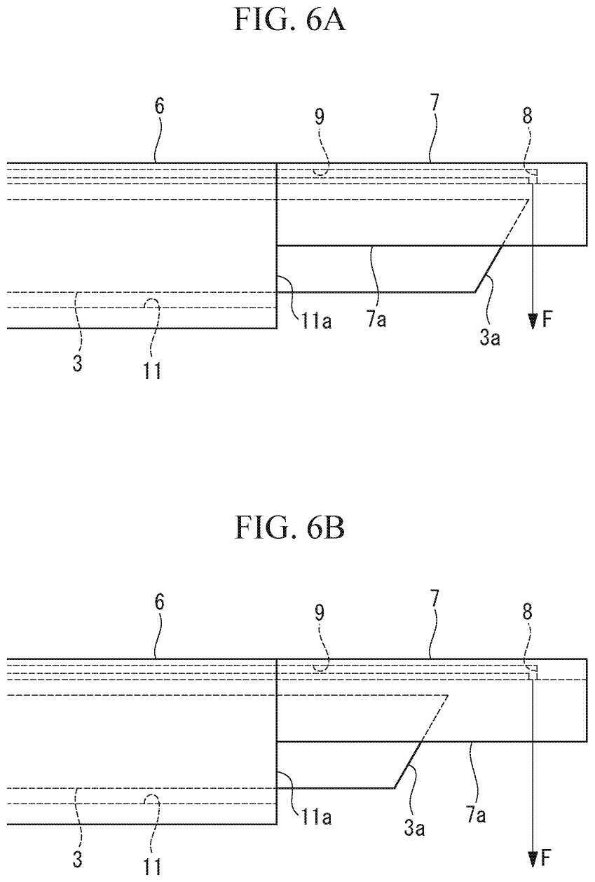

Although the endoscope 2 has been described as a forward-viewing endoscope in this embodiment, instead, as shown in FIGS. 6A and 6B, the endoscope 2 may be an oblique-viewing endoscope, in which the observation window 5 is provided in the distal end face 3a that is inclined with respect to the longitudinal direction.

In the case of the oblique-viewing endoscope 2, because the bending portion 4 does not need to be bent when the heart A is observed from above, it is possible to ensure a larger distance between the heart A and the distal end face 3a than in the forward-viewing endoscope 2.

When the heart A is to be cleaned, instead of operating the bending portion 4, the operation of moving the insertion section 3 toward the proximal end, to a position where the distal end face 3a does not interfere with the cleaning fluid F jetted from the nozzle 8, is performed, as shown in FIG. 6B. This makes it possible to clean the observation area of the heart A with the cleaning fluid F while the observation area of the heart A is observed by using the endoscope 2.

As has been described above, in the case of the oblique-viewing endoscope 2, because the cleaning target is switched by moving the insertion section 3 in the longitudinal direction, the fixing portion 10 may be omitted.

Alternatively, instead of the fixing portion 10, it is possible to provide a rotation restricting portion that restricts rotation of the insertion section 3 inside the endoscope channel 11, while allowing movement of the insertion section 3 in the longitudinal direction inside the endoscope channel 11. The rotation restricting portion restricts rotation of the insertion section 3 inside the endoscope channel 11 to hold the insertion section 3 in an orientation in which the distal end face 3a is oriented towards the cutaway portion 7a. The rotation restricting portion includes, for example, a radially projecting projection provided on the outer circumferential surface of the insertion section 3, and a groove provided in the inner circumferential surface of the endoscope channel 11 so as to extend in the longitudinal direction to guide the projection inserted therein. It is also possible that the projection is provided on the inner circumferential surface of the endoscope channel 11, and the groove is provided in the outer circumferential surface of the insertion section 3.

In this embodiment, the sheath body 6 may further have a treatment tool channel through which a treatment tool is inserted.

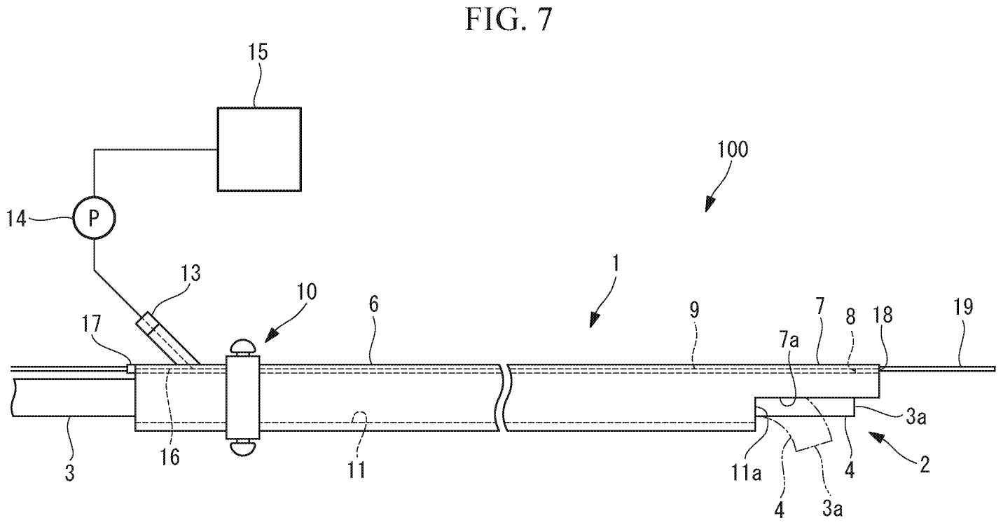

Although the treatment tool channel may be formed completely independent of the fluid channel 9, a part of the fluid channel 9 may be used as the treatment tool channel, as shown in FIG. 7.

The sheath body 6 in FIG. 7 further has a treatment-tool insertion port 17 provided in the proximal end face of the sheath body 6, and a treatment tool channel 16 extending in the longitudinal direction from the treatment-tool insertion port 17 toward the distal end and communicating with the fluid channel 9 at an intermediate position of the fluid channel 9. The fluid channel 9 extends to a treatment tool opening 18, which is provided in the distal end face of the projecting portion 7 and into/from which the treatment tool can be retracted/projected. This configuration makes it possible to insert the treatment tool into the pericardial space through the treatment-tool insertion port 17, the treatment tool channel 16, the fluid channel 9, and the treatment tool opening 18. It is desirable that valves that prevent leakage of the cleaning fluid F while allowing the treatment tool to move in the longitudinal direction be provided at the treatment-tool insertion port 17 and the treatment tool opening 18.

FIG. 7 shows a guide wire 19, serving as an example treatment tool. As shown in FIG. 7, by projecting the guide wire 19 from the distal end of the projecting portion 7, it is possible to lift the pericardium B with the guide wire 19 also on the distal-end side of the projecting portion 7 to produce a larger space S around the distal end face 3a of the insertion section 3. Another type of treatment tool may be used instead of the guide wire 19.

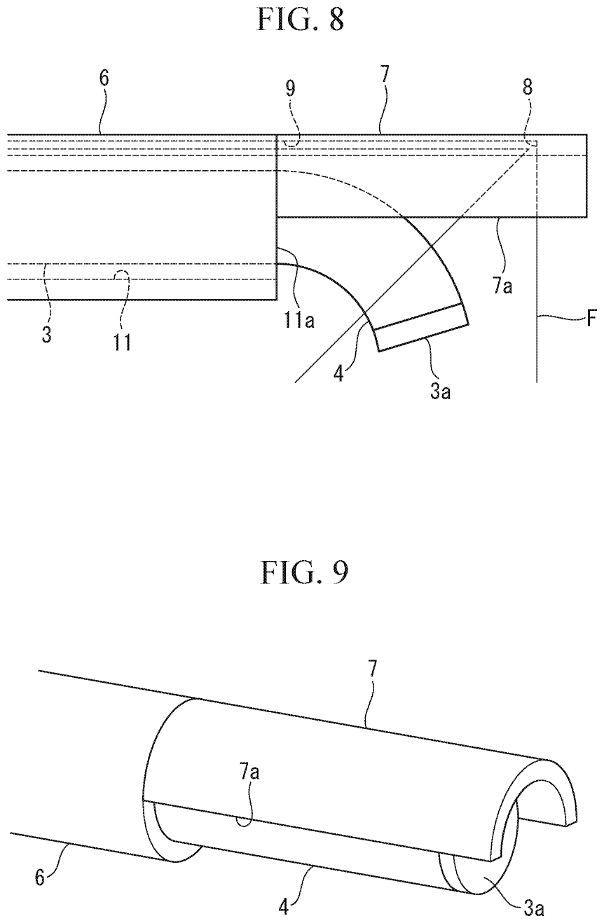

Although the inner surface of the nozzle 8 is formed perpendicular to the longitudinal direction of the endoscope channel 11 in this embodiment, instead, the inner surface of the nozzle 8 closer to the proximal end may be inclined toward the proximal end, as shown in FIG. 8.

This configuration allows the cleaning fluid F to be jetted from the nozzle 8 also in an oblique direction toward the proximal end. Accordingly, in a state in which the bending portion 4 is bent, the cleaning fluid F is simultaneously sprayed over the heart A and the observation window 5, thus simultaneously cleaning both the observation area of the heart A and the observation window 5.

Although the endoscope 2 and the sheath body 6 are separate components in this embodiment, instead, as shown in FIG. 9, the endoscope 2 and the sheath body 6 may be integrally formed by fixing the outer circumferential surface of the insertion section 3 and the inner circumferential surface of the endoscope channel 11 to each other. The endoscope 2 and the sheath body 6 are fixed such that the distal end face 3a of the insertion section 3 is adjacent to the nozzle 8 in the radial direction and such that the cutaway portion 7a is provided in the direction in which the bending portion 4 can be bent.

This configuration makes it possible to eliminate the need for the task of fixing the insertion section 3 and the sheath body 6. Furthermore, because there is no need to provide, inside the endoscope channel 11, a clearance for enabling the insertion section 3 to move forward/backward and rotate inside the endoscope channel 11, the diameter of the sheath body 6 can be reduced.

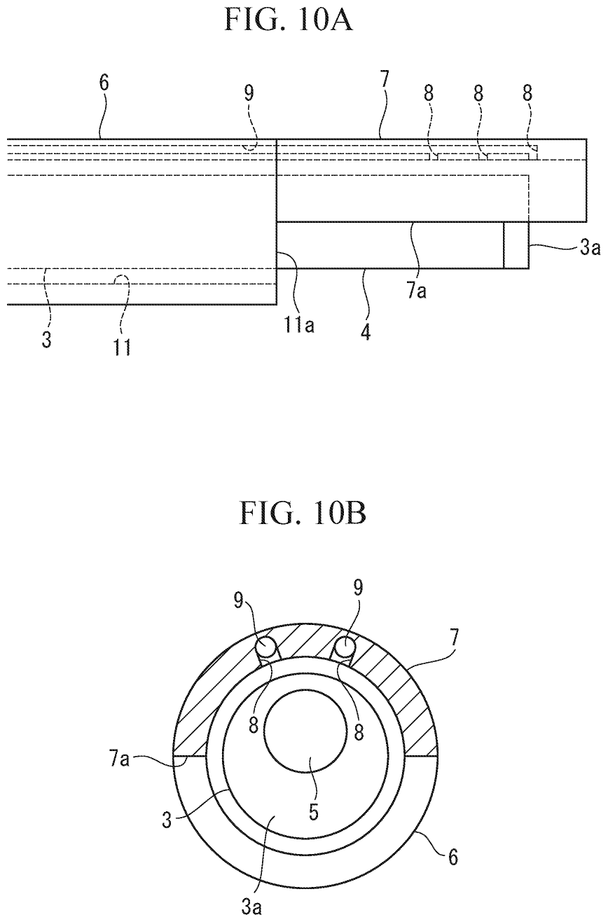

Although there is only one nozzle 8 provided in this embodiment, instead, multiple nozzles 8 may be provided. The multiple nozzles 8 may be arranged either in the longitudinal direction of the projecting portion 7, as shown in FIG. 10A, or in the circumferential direction of the projecting portion 7, as shown in FIG. 10B.

Providing multiple nozzles 8 like this makes it possible to clean a larger area of living body tissue. When the multiple nozzles 8 are arranged in the longitudinal direction, as shown in FIG. 10A, it is possible to also clean the adherent adhered to the side surface of the distal end portion of the insertion section 3.

The fluid channel 9 may be provided for each nozzle 8. By doing so, even if any one of the fluid channels 9 and nozzles 8 is clogged, the observation window 5 and the heart A can be cleaned by using another fluid channel 9 and nozzle 8.

Alternatively, it is possible to provide one common fluid channel 9 for the multiple nozzles 8.

The above-described embodiment leads to the following invention.

A first aspect of the present invention is an endoscope sheath including: a tubular sheath body having a penetrating endoscope channel, through which an endoscope is inserted, extending in the longitudinal direction; a projecting portion extending in the longitudinal direction from the distal end of the sheath body to a position beyond the distal end portion of the endoscope projecting from the distal end opening of the endoscope channel, the projecting portion being provided at a portion in the circumferential direction of the endoscope channel, on the outer side of the endoscope channel in the radial direction; a nozzle from which cleaning fluid is jetted radially inward of the sheath body, onto the distal end face of the distal end portion of the endoscope projecting from the distal end opening of the endoscope channel, the nozzle being provided in the distal end portion of the projecting portion; and a fluid channel through which the cleaning fluid is supplied to the nozzle, the fluid channel being provided in the sheath body and the projecting portion so as to extend in the longitudinal direction. The side opposite the nozzle with the endoscope channel therebetween in the radial direction is open at the distal end of the sheath body.

In the first aspect of the present invention, the projecting portion having the nozzle is provided at the distal end of the sheath body, at a portion in the circumferential direction, and the side opposite the nozzle is open. Accordingly, by disposing the sheath body such that the open portion is oriented towards an observation-target tissue and by causing the distal end portion of the endoscope inserted through the endoscope channel in the sheath body to project from the distal end opening of the endoscope channel to the projecting portion, it is possible to observe the observation-target tissue with the endoscope. When a user intends to clean an observation area of the observation-target tissue, by disposing the endoscope so as not to be located between the nozzle and the observation area of the observation-target tissue and supplying the cleaning fluid to the nozzle through the fluid channel, it is possible to spray the cleaning fluid jetted from the nozzle onto the observation area of the observation-target tissue to clean the observation area.

Furthermore, when the user intends to clean the observation window in the distal end face of the endoscope, by disposing the distal end face of the endoscope at a position radially adjacent to the nozzle and supplying the cleaning fluid to the nozzle through the fluid channel, it is possible to spray, from the nozzle, the cleaning fluid onto the distal end face of the endoscope to clean the observation window.

In the first aspect above, the projecting portion may have a plurality of the nozzles.

This configuration makes it possible to jet the cleaning fluid over a wide area and, thus, to clean larger areas of the distal end face of the endoscope and the observation-target tissue.

In the first aspect above, the endoscope sheath may further includes a treatment tool channel through which a treatment tool is inserted, the treatment tool channel extending in the longitudinal direction from the proximal end portion of the sheath body and communicating with the fluid channel at an intermediate position of the fluid channel in the longitudinal direction. The fluid channel may allow the treatment tool to pass therethrough and may extend to a treatment tool opening that is open in the distal end of the projecting portion and into/from which the treatment tool is retracted/projected.

This configuration makes it possible to allow the treatment tool inserted through the fluid channel through the treatment tool channel to project from the treatment tool opening provided at the distal end of the projecting portion. By using the fluid channel also as the channel for the treatment tool like this, the diameter of the sheath body can be made smaller than that in the case where the treatment tool channel that is separate from the fluid channel is provided over substantially the overall length of the sheath body.

In the first aspect above, the projecting portion may have a substantially partial cylindrical shape having, in a portion in the circumferential direction, a cutaway portion removed in the longitudinal direction.

With this configuration, because the distal end portion of the endoscope is covered by the projecting portion on the side opposite to the cutaway portion, it is possible to protect the distal end portion of the endoscope from the surrounding living body tissue.

A second aspect of the present invention is an endoscope system including: the endoscope sheath according to the above-described aspect; and an endoscope that is inserted in the longitudinal direction through the endoscope channel in the endoscope sheath.

In the second aspect, the endoscope may be movable in the longitudinal direction inside the endoscope channel.

This configuration makes it possible to switch the cleaning target to be cleaned with the cleaning fluid between the observation window and the observation area of the observation-target tissue, by moving the endoscope in the longitudinal direction between the position where the distal end face of the endoscope is adjacent to the nozzle and the position where the distal end face of the endoscope is located closer to the proximal end than the nozzle is.

In the second aspect above, the endoscope system may further include a position restricting portion that restricts the relative positions, in the longitudinal direction, of the endoscope sheath and the endoscope to the positions where the distal end of the endoscope is aligned with the nozzle in the radial direction.

This configuration makes it possible to restrict the relative positions of the endoscope sheath and the endoscope such that the distal end face of the endoscope is located at a position suitable for cleaning the observation window.

In the second aspect above, the endoscope may have a bendable bending portion at the distal end portion, and a rotation restricting portion that restricts rotation of the endoscope about the longitudinal axis in the endoscope channel to enable the bending portion to bend to the side opposite to the nozzle may be provided.

This configuration makes it possible to restrict the orientation of the endoscope about the longitudinal axis in the endoscope channel, such that the bending portion of the endoscope is bent toward the observation-target tissue.

In the second aspect above, the endoscope sheath and the endoscope may be integrally formed.

This configuration makes it possible to eliminate the need for attaching the endoscope sheath to the endoscope.

### REFERENCE SIGNS LIST

1 endoscope sheath
2 endoscope
3 insertion section
3a distal end face
4 bending portion
5 observation window
6 sheath body
7 projecting portion
7a cutaway portion
8 nozzle
9 fluid channel
10 fixing portion (position restricting portion, rotation restricting portion)
11 endoscope channel
13 supply port
14 pump
15 fluid source
16 treatment tool channel
17 treatment-tool insertion port
18 treatment tool opening
19 guide wire (treatment tool)
100 endoscope system
F cleaning fluid
A heart
B pericardium

1. An endoscope sheath comprising:
a tubular sheath body having a penetrating endoscope channel, through which an endoscope is inserted, extending in a longitudinal direction;
a projecting portion extending in the longitudinal direction from a distal end of the sheath body to a position beyond a distal end portion of the endoscope projecting from a distal end opening of the endoscope channel, the projecting portion being provided at a portion in a circumferential direction of the endoscope channel, on an outer side of the endoscope channel in a radial direction;
a nozzle from which cleaning fluid is jetted radially inward of the sheath body, onto a distal end face of the distal end portion of the endoscope projecting from the distal end opening of the endoscope channel, the nozzle being provided in a distal end portion of the projecting portion;
a fluid channel through which the cleaning fluid is supplied to the nozzle, the fluid channel being provided in the sheath body and the projecting portion so as to extend in the longitudinal direction; and
a treatment tool channel through which a treatment tool is inserted, the treatment tool channel extending in the longitudinal direction from a proximal end portion of the sheath body and communicating with the fluid channel at an intermediate position of the fluid channel in a longitudinal direction,
wherein a side opposite the nozzle with the endoscope channel therebetween in the radial direction is open at the distal end of the sheath body, and
wherein the fluid channel allows the treatment tool to pass therethrough and extends to a treatment tool opening that is open in a distal end of the projecting portion and into/from which the treatment tool is retracted/projected.

2. The endoscope sheath according to claim 1, wherein the projecting portion has a plurality of the nozzles.

3. The endoscope sheath according to claim 1, wherein the projecting portion has a substantially partial cylindrical shape having, in a portion in a circumferential direction, a cutaway portion removed in a longitudinal direction.

4. An endoscope system comprising:
the endoscope sheath according to claim 1; and
an endoscope that is inserted in a longitudinal direction through the endoscope channel in the endoscope sheath.

5. The endoscope system according to claim 4, wherein the endoscope is movable in a longitudinal direction inside the endoscope channel.

6. The endoscope system according to claim 5, further comprising a screw that restricts the relative positions, in the longitudinal direction, of the endoscope sheath and the endoscope to the positions where a distal end of the endoscope is aligned with the nozzle in the radial direction.

7. The endoscope system according to claim 5, wherein
the endoscope has a bendable bending portion at the distal end portion,
a projection is provided on one of an inner circumferential surface of the endoscope channel and an outer circumferential surface of the endoscope,
a groove is provided on the other of the inner circumferential surface of the endoscope channel and the outer circumferential surface of the endoscope, and
rotation of the endoscope about a longitudinal axis in the endoscope channel is restricted by the groove guiding the projection to enable the bending portion to bend to the side opposite to the nozzle.

8. The endoscope system according to claim 4, wherein the endoscope sheath and the endoscope are integrally formed.
